(19) United States
(12) Patent Application Publication
(10) Pub. No.: US 2016/0317322 A1
(43) Pub. Date: Nov. 3, 2016

(54) IMPLANT AND METHODS FOR PRODUCING AN IMPLANT

(71) Applicant: Warsaw Orthopedic, Inc, Warsaw, IN (US)

(72) Inventors: Armin Studer, Cham (CH); Jorge Garcia Forgas, Embrach (CH); Armando Salito, Wohlen (CH)

(21) Appl. No.: 15/207,251

(22) Filed: Jul. 11, 2016

Related U.S. Application Data
(63) Continuation of application No. 13/641,024, filed on Oct. 12, 2012, now Pat. No. 9,414,923.

(51) Int. Cl. A61F 2/44; A61F 2/32; A61F 2/40; A61F 2/38

(52) U.S. Cl. CPC A61F 2/4455; A61F 2/38; A61F 2/32; A61F 2/40; A61F 2002/30013

(57) ABSTRACT

An implant is provided with a main body (1) and with a double coating applied to at least one surface section of the main body (1), wherein the double coating comprises an adhesion promoter layer (2), applied directly to the at least one surface section of the main body, and of an osteointegrative layer (3) covering the adhesion promoter layer (2). The layers (2;3) consist of pure titanium, wherein the adhesion promoter layer (2) has a thickness of 2-6 μm, in particular a thickness of 3-5 μm, and the osteointegrative layer (3) has a thickness of 50-70 μm, in particular of 55-65 μm. Moreover, the osteointegrative layer (3) has a porosity of 70-90% and a roughness R_{z} of at least 45 μm. A related method is also provided for producing such an implant.

### RELATED APPLICATIONS

This application claims the benefit of International Application No. PCT/EP2011/055710 filed Apr. 12, 2011, now publication No. WO 2011/128334, which claims priority to German Patent Application No. DE 102010010599, filed Apr. 15, 2010. The contents of each of these priority applications are incorporated herein by reference, in their respective entireties.

### TECHNICAL FIELD

The present disclosure generally relates to medical devices, systems and methods for the treatment of musculoskeletal disorders, and more particularly, to an implant having a main body and a double coating applied to at least one surface section of the main body. Furthermore, a method is provided for producing such an implant.

### BACKGROUND

As is known, many implants, prostheses or endoprostheses are produced of polyether ether ketone (PEEK). Compared to metallic materials, this material has the advantage that the Elastic modulus of PEEK corresponds more to the Elastic modulus of cortical bones than metallic materials could ever achieve. Moreover, PEEK is permeable to X-rays, as a result of which the physician can observe bone integration of, for example, vertebral cages by means of corresponding radiograms during follow-up treatment. This would not be possible with a titanium cage.

However, for some time, implants and the like which are produced of PEEK have become subject to criticism. It could be observed that the human or animal bone does not completely adhere to the implant and grow into the implant, respectively. The bones rather form a seam on the surface of the PEEK material. In case that such a seam formation can be discovered on an X-ray image, this means that bone adhesion has not happened and that there is no sufficient stability regarding the inserted implant.

As a result of this, the implant either has to be removed and be replaced by a new one, or the implant has to be firmly fixed to the bone by means of other surgical methods. Another surgery is associated with additional stress, pain, and corresponding surgical risks for the patient.

Metallic material, particularly titanium, fulfills optimal conditions regarding growth into animal or human bone structures. It is proven that the bones adhere to the titanium, and, provided that the surface is accordingly designed, the bone can also grow into the microstructures of titanium materials.

Thus for a long time, there have been attempts to develop coatings and implant materials, respectively, such that, on the one hand, an improved bioactive surface layer and a related grow-into ability for animal or human bones is achieved, and, on the other hand, good Elastic moduli, as already realized through the usage of PEEK materials, can be obtained.

In EP 1372749 B1, a bioactive surface layer for implants and prostheses is disclosed, wherein the implant can consist of PEEK. A variable part of the surface layer consists of calcium phosphate phases, wherein the CA-ions and PO₄-ions embedded in the surface layer are completely spread over a metal oxide layer. The metal oxide is titanium oxide, for instance. Furthermore, an additional coating of the surface layer with hydroxylapatite is described. Such hydroxylapatite coatings of implants are common methods to ensure improved growth of bone structures into the implant.

However, tests of the tensile strength values to be achieved and of the shearing forces to be resisted of common implants have fueled the desire for improved implants regarding the two values, but wherein the implants should also grow into animal or human bone structures in such a good manner, as is the case with an implant coating with hydroxylapatite, for instance.

Due to the aforementioned, the task of the embodiments presented here therefore is to provide an improved implant and method, comprising a coating which can be realized cost-efficiently, has improved tensile strength values, and which can be loaded with higher shearing forces. Furthermore, a method is provided, with the help of which a quick and cost-efficient production of a coated implant can be realized.

### SUMMARY

### BRIEF DESCRIPTION OF THE DRAWINGS

The present disclosure will become more readily apparent from the specific description accompanied by the following drawings, in which:

FIG. 1 shows a cross-sectional view through an implant according to one embodiment; and

FIG. 2 shows a top view of a vertebral body implant according to one embodiment.

### DETAILED DESCRIPTION

In one embodiment, an implant comprises a main body, wherein a double coating is partially applied to the surface of the main body. It is conceivable to apply the double coating to the entire surface of the implant main body, but due to cost and dimensioning reasons regarding the overall thickness of an implant, merely individual surface sections or only one surface section of the main body should be provided with said coating. Prior to the production method, the surface sections to be coated have to be calculated, namely depending on the implant to be produced and the size of the implant.

For instance, the implant can be vertebral cages, knee and hip prostheses and endoprostheses, respectively, bone prostheses or artificial shoulder joints. The implant embodiments herein are important particularly in matters of cementless prosthetics, but also in dental prosthetics.

When determining the surface sections, the size of the patient undergoing surgery is also important. For instance, implants, prostheses, and endoprostheses have different dimensions, depending on gender, size or weight of the patient. In case of implants for insertion in animal bodies, there are also often different implant sizes provided. The surface section(s) to be coated has to be dimensioned depending on the later load, tension, and the shearing forces applied to the implant in the inserted state. For instance, it can be sufficient in some cases, to determine a single continuous surface section, however, it is also conceivable to define several surface sections which are spaced apart from each other.

In the inserted state, i. e. when the implant is inserted in the human or animal body, the at least one surface section to be coated of the implant main body is directed to a bone of the body. With the double coating to be applied, the surface section(s) form the surface(s) of the implant adjacent to the bone.

First of all, said double coating consists of an interlayer and an adhesion promoter layer, respectively, which is directly applied to the determined surface sections and the determined surface section of the implant main body, respectively.

This adhesion promoter layer is completely covered with an osteointegrative layer. Both layers, i.e. the adhesion promoter layer and the osteointegrative layer, consist of pure titanium. F or the adhesion promoter layer, a layer thickness of 2-6 μm, in particular a thickness of 3-5 μm, is aimed for. The osteointegrative layer comprises a layer thickness of 50-70 μm, in particular of 55-65 μm. Provided that several surface sections of the implant main body to be coated are determined, the sections separated from each other respectively have to be provided with the same layer thickness regarding the adhesion promoter layer and the osteointegrative layer.

According to one embodiment, the osteointegrative layer has a porosity of 70-90% and a roughness R_{z} of at least 45 μm. I. e., the value of roughness R_{z} amounts to at least 45 μm, but can be greater, e. g. 55 μm.

The main body of the implant preferably is produced of polyether ether ketone (PEEK), wherein the main body can also consist of other synthetic material, such as polyoxymethylene (POM), polyaryletherketone (PAEK), polyetherimide (PEI), polymethylpentene (PMP), polyethersulfone (PES), polysulfone (PSU), polymethyl methracylate (PMMA) or polyethylene terephthalate (PETP).

Due to the fact that, as described, the osteointegrative layer is produced of pure titanium and that, as experience has shown, implant coatings of a different material tend to chip or break off, for instance, it is the task of the adhesion promoter layer in accordance with one embodiment, to establish a stable connection between the osteointegrative layer and the implant main body. Particularly through the selected porosity of 70-90& regarding the osteointegrative layer, such a layer would, directly applied to the previously defined surface sections of the PEEK implant main body, not achieve a sufficient and permanent adhesion, respectively.

Due to the fact that the adhesion promoter layer also consists of pure titanium, adhesion of the osteointegrative layer to the adhesion promoter layer is implemented without problems. The adhesion promoter layer comprises a high density, to which the final porous layer—the osteointegrative layer—is applied.

The porous layer, also called porous coating, ensures excellent instant implant stability as well as an outstanding adhesion of the animal or human bone to the implant. Even after an already longer implantation time of several years, a symptom of tiring concerning the stability of the implant can be observed.

In a particularly preferred embodiment of the implant, the adhesion promoter layer comprises a thickness of substantially 3 μm, wherein substantially in this case means that deviations of ±0.5 μm are possible.

The osteointegrative layer preferably has a layer thickness of substantially 60 μm. With this layer thickness, deviations of ±3 μm lie within the scope of possibility.

With the mentioned layer thicknesses, particularly good results can be achieved regarding the tensile strength values and the possibly applied shearing forces.

Furthermore, it is pointed out that an osteointegrative layer having a porosity of 80%±5% is particularly preferred.

The method for producing an implant/a coated implant initially comprises the step of applying a mask to the implant main body. The mask comprises dimensions and recesses, such that the at least one calculated and defined surface section is exposed, and the not to be coated surface sections are covered with the mask. The mask provided with recesses is preferably produced of silicone.

Afterwards, an application of the at least one surface section to be coated with a blasting material is implemented. This means that the exposed and non-covered surface of the implant main body is applied, i. e. blasted with the blasting material.

The blasting material preferably is special fused alumina, which causes roughening of the surface section when applied to the PEEK main body. This process is implemented at a pressure of 1 to 3 bar, preferably at 2 bar.

The roughening causes an improved adhesion of the adhesion promoter layer applied to the at least one surface section of the implant main body in a subsequent step. The application of the adhesion promoter layer is implemented by means of a vacuum-based coating method, i. e. with a PVD method. In doing so, a layer having a thickness of 2-6 μm, in particular a thickness of 3-5 μm, of dense titanium material is applied to the surface section(s).

In another method step, this adhesion promoter layer or interlayer is provided with a final osteointegrative layer consisting of pure titanium. A layer having a thickness of 50-70 μm, in particular of 55-65 μm, is applied, which has a porosity of 70-90% and a roughness R_{z} of at least 45 μm.

The porous coating, for instance, can be applied to the adhesion promoter layer by means of an electron melting method. In this case, sintering powder and titanium powder, respectively is applied layerwise to the adhesion promoter layer, and fused together and subsequently cooled according to the respective dimensions of the cross-sectional layer by means of energy application through a radiation source. The energy output by the radiation source only has an impact on the powder particles which are to be solidified, therefore representing a material particle of the later implant. Subsequently, the next cross-sectional layer is applied to the already fused material and is in turn melted by means of energy application. The process is implemented layer after layer in the vertical direction.

The electron melting method is particularly suitable to achieve the desired porosity of 70-90% of the osteointegrative layer.

The following results could be achieved with implemented tests of PEEK main bodies having a titanium double coating consisting of an adhesion promoter layer and an osteointegrative layer:

TABLE 1
Tensile strength test according to ASTM F1044

| | Tensile strength test according |
|---|---|
| | to ASTM F1147 |
| test run 1 (6 samples) | X = 59.7 MPa σ = 4.8 MPa |
| test run 2 (5 samples) | X = 56.4 MPa σ = 4.8 MPa |
| test run 3 (5 samples) | X = 35.6 MPa σ = 6.7 MPa |
| test run 4 (5 samples) | X = 41.5 MPa σ = 5.3 MPa |

TABLE 2
Shearing force test according to ASTM F1044

| | Shearing force test according |
|---|---|
| | to ASTM F1044 |
| test run 1 (5 samples) | X = 37.8 MPa σ = 1.7 MPa |
| test run 2 (5 samples) | X = 38.2 MPa σ = 4.3 MPa |
| test run 3 (5 samples) | X = 29.1 MPa σ = 2.8 MPa |

Hereafter, selected embodiments are explained in more detail with reference to the attached schematic drawings.

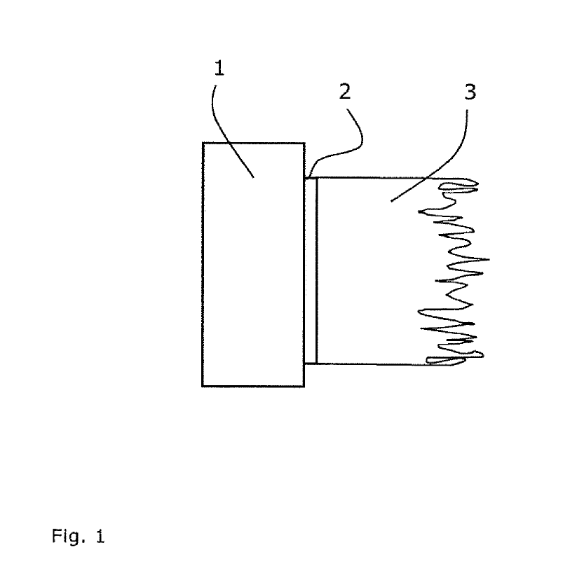

As shown in FIG. 1, an adhesion promoter layer 2 is applied to a main body 1 of the implant first of all. This adhesion promoter layer 2 is located on at least one surface section of the main body 1, wherein the number and the size of the surface sections are inter alia depending on the dimensions of the implants and the size, the weight, and the gender of the patient.

Prior to applying the adhesion promoter layer 2 to the surface section(s), the main body 1 is covered with a mask. This mask, for instance, consists of silicone and defines the surface section to be coated by means of recesses, i. e. the surface section is not covered by the silicon material of the mask.

Subsequently, the surface section is applied with a blasting material, preferably special fused alumina, at a pressure of 2 bar, in order to cause roughening of the surface section.

What follows is the coating of the roughened surface section with a very dense adhesion promoter layer 2 of pure titanium. This layer 2 comprises a thickness of 3 μm, wherein the application process is implemented by means of a PVD method.

In a final method step, the surface section provided with an adhesion promoter layer 2 is provided with an osteointegrative layer 3. This layer comprises a thickness of substantially 60 μm at a porosity of 80% and a roughness R_{z} of 50 μm. The application of the osteointegrative layer 3 is implemented by means of an electron beam melting method on the basis of titanium powder.

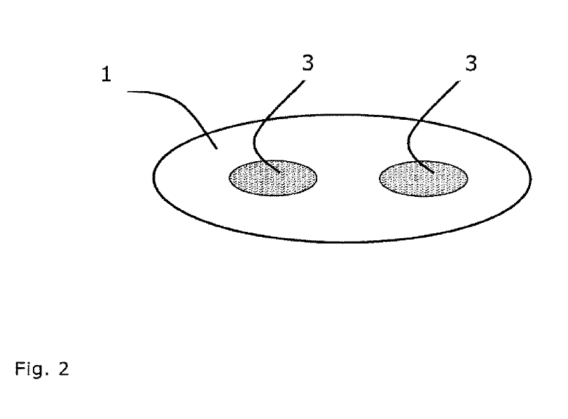

In FIG. 2, a vertebral body implant having a main body 1 is schematically illustrated. It is obtainable from the top view that two surface sections of the main body comprise a coating. Consequently, the osteointegrative layer 3 directed to the bone of the human or animal patient consists of two individual areas which are spaced apart from each other. The silicon mask used in the method for applying the double coating to the implant comprises two recesses in the form of both individual areas of the osteonintegrative layer 3.

It should be noted that, when defining several surface sections of the main body, the layer thicknesses of the respective adhesion promoter layers 2 and osteointegrative layers 3 correspond, i. e. comprise substantially the same thickness values. However, deviations of ±0.5 μm do not play an important role.

1-5. (canceled)

6. Method for manufacturing an implant, comprising:
applying a mask to a main body of an implant so as to define at least one surface section of the main body to be coated;
blasting the at least one surf ace section with a blasting material;
applying an adhesion promoter layer comprising titanium having a thickness of 2-6 μm using a vacuum-based coating method to the at least one defined surface section: and applying an osteointegrative layer comprising titanium to the applied adhesion promoter layer, the osteointegrative layer having a thickness of 50-70 μm a porosity of 70-90%, and a roughness R_{z} of at least 45 μm.

7. Method according to claim 6, wherein the blasting material comprises fused alumina.

8. Method according to claim 6, wherein the blasting step comprises blasting with a pressure of 1-3 bar.

9. Method according to claim 6, wherein the mask comprises a silicone mask applied to the main body.

10. Method according to one of claims 6, wherein the osteointegrative layer is applied to the adhesion promoter layer via electron beam melting.

11. Method according to claim 1, wherein the adhesion promoter layer is applied having a thickness of 3-5 μm.

12. Method according to claim 1, wherein the osteointegrative layer is applied having a thickness of 55-65 μm.

13. Method according to claim 8, wherein the blasting step comprises blasting with a pressure of 2 bar.

14. The method for producing a coated implant comprising PEEK and having a main body with surface sections, comprising:
exposing at least one calculated and defined surface section to be coated;
covering the surface sections that will not be coated;
applying blasting material to the at least one calculated and defined surface section; and
applying an adhesion promoter layer having a thickness of 2 μm to 6 μm to the at least one calculated and defined surface section.

15. The method of claim 14, further comprising providing an osteointegrative layer comprising titanium with the adhesion promoter layer.

16. The method of claim 15, wherein the osteointegrative layer is applied having a thickness of 50 μm to 70 μm.

17. The method of claim 16, wherein the osteointegrative layer is applied having a thickness of 55 μm to 65 μm.

18. The method of claim 14, wherein the osteointegrative layer has a porosity of 70% to 90%.

19. The method of claim 14, wherein the adhesion promoter layer is applied using a vacuum-based coating method,

20. The method of claim 14, further comprising applying a porous coating to the adhesion promoter layer by means of an electron melting method.
